(19) United States
(12) Patent Application Publication
(10) Pub. No.: US 2019/0365609 A1
(43) Pub. Date: Dec. 5, 2019

(54) METHOD AND APPARATUS FOR A NIPPLE CUSHION

(71) Applicant: BeauGen, LLC, Orlando, FL (US)

(72) Inventors: Tu-Hien Le, Orlando, FL (US); Christopher Chang, Orlando, FL (US); Carlos Vega, Orlando, FL (US)

(73) Assignee: BeauGen, LLC

(21) Appl. No.: 15/994,881

(22) Filed: May 31, 2018

(51) Int. Cl. A61J 13/00

(52) U.S. Cl. CPC A61J 13/00; A61M 1/06

(57) ABSTRACT

A nipple cushion formed as part of an insert for a breast-receiving funnel-shaped membrane of a breast pump. The nipple cushion may include an adhesive to form a temporary bond to any type of a breast pump cushion. Such a nipple cushion of the present invention absorbs any friction created by the movement between the breast pump and the user's breast, with the aid of the material that constitutes the nipple cushion.

### FIELD OF THE INVENTION

The present invention generally relates to an insert for a breast pump, and in particular the invention relates to a breast nipple cushion adapted to protect a user's breast nipple and areola during the pumping of breast milk.

### BACKGROUND OF THE INVENTION

Breast pumps are well known devices for extracting milk from the breast of a user. A breast pump may be used if the baby is not itself able to extract the milk, or if the mother is separated from the baby, for example if away from the baby at work. The use of a breast pump to extract milk may also be used to stimulate and increase milk production in women with a low milk supply.

Conventional breast pumps are vacuum-based, as such, a breast pump makes use of a vacuum to induce milk extraction from a nursing mother's breast. The pumping action of the device draws the milk from the nipple to a collection vessel, and the pressure and/or frequency may be adjusted to the preferences of the mother. Therefore, the adjustability of the breast pump to the preference of the mother is limited.

In the field of breast pumps for expressing breast milk, the typical breast pump's funnel-shaped breast cushions are generally constructed from a rigid plastic and/or Silicone material. One of the problems with the breast pumps available nowadays, is that users are known to experience some form of discomfort or difficulty when using such a breast pump. The rigidity of the breast cushion's material tends to generate various levels of friction and compression that can lead to discomfort in the form sores, bruises, cracks, blisters, swollen, or bleeding skin or tissue around the user's areola and nipple.

Despite many breast pump manufacturers' attempts to solve this problem, there has yet to be a universal solution that can be used across all brands, parts, and sizes to reduce discomfort for the breast pump use.

As can be seen throughout this application, there is a need for a nipple cushion usable by breastfeeding mothers that facilitates the prevention of any form of discomfort or injury to their nipples and areolas when using a breast pump to extract breast milk. Compatibility with a variety pumps would also be desirable aspect.

Despite the various-sized breast cushion pumps, and accessories that current manufactures produce, not all women may be able to achieve a proper fit. In the event that a breast cushion is too big for the woman using it, milk may not be able to be extracted because proper suction or enough pressure is achieved to allow a letdown of breastmilk. Conversely, in the event that the breast cushion is too small for the woman using it, increased friction may be experienced between the areola/nipple and the breast cushion creating more discomfort and possibly pain. Additionally, a breast cushion that is too small can block milk ducts, and prevent the letdown of the breastmilk.

### SUMMARY OF THE INVENTION

The objective of the present invention is to present a breast pump insert which considerably eases and possibly overcomes matters of discomfort to a user. Specifically, the apparatus of the present invention reduces or possibly eliminates friction felt by users using any of the nipple cushions that exist today.

In a first embodiment the present invention provides a breast pump which substantially alleviates or overcomes the problems mentioned above and aids in the extraction of breast milk from a user's breast in a fashion which is safer and less injurious.

In a second embodiment, the present invention provides nipple cushion. Such a nipple cushion takes a shape of a funnel. The nipple cushion of the present invention is supple and may take the form of the funnel-shaped breast cushion that is part of breast pumps available in the market.

Preferably, the supple nipple cushion is configured to deform towards a user's areola and nipple, reducing any frictional forces that may create soreness of the user's nipple and areola.

The fourth embodiment may further comprise a method of manufacturing the nipple cushions via the material being hand-poured, injection molding, or by additive manufacturing of materials including thermoplastic elastomer, silicone, rubber, natural rubber, latex or other rubber-like material.

### BRIEF DESCRIPTION OF THE DRAWINGS

In order to facilitate a full understanding of the present invention, reference is now made to the accompanying drawings, in which like elements are referenced with like numerals. These drawings should not be construed as limiting the present invention, but are intended to be exemplary and for reference.

FIG. 1 provides a perspective view of a nipple cushion in accordance with an embodiment of the present invention.

FIG. 2 provides a cross-sectional view of a nipple cushion in accordance with an embodiment of the present invention.

FIG. 3 provides a top plan view of a nipple cushion in accordance with an embodiment of the present invention.

FIG. 4 provides an exploded view of a nipple cushion when used with a breast pump in accordance with an embodiment of the present invention.

FIG. 5 provides a frontal view of a user of the nipple cushion of the present invention as it would be applied to a breast

FIG. 6 provides a Shore table of hardness and softness of materials, showing the hardness and softness of materials used in the manufacture of the nipple cushion in accordance with an embodiment of the present invention.

### DETAILED DESCRIPTION OF THE INVENTION

The present invention will now be described in more detail with reference to exemplary embodiments as shown in the accompanying drawings. While the present invention is described herein with reference to the exemplary embodiments, it should be understood that the present invention is not limited to such exemplary embodiments. Those possessing ordinary skill in the art and having access to the teachings herein will recognize additional implementations, modifications, and embodiments, as well as other applications for use of the invention, which are fully contemplated herein as within the scope of the present invention as disclosed and claimed herein, and with respect to which the present invention could be of significant utility.

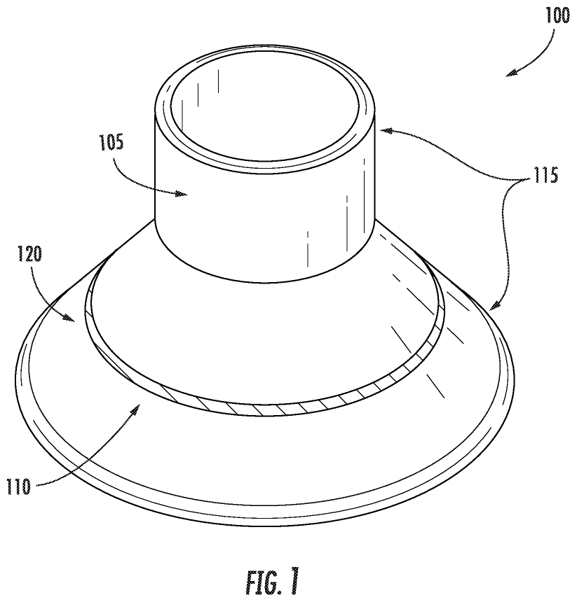

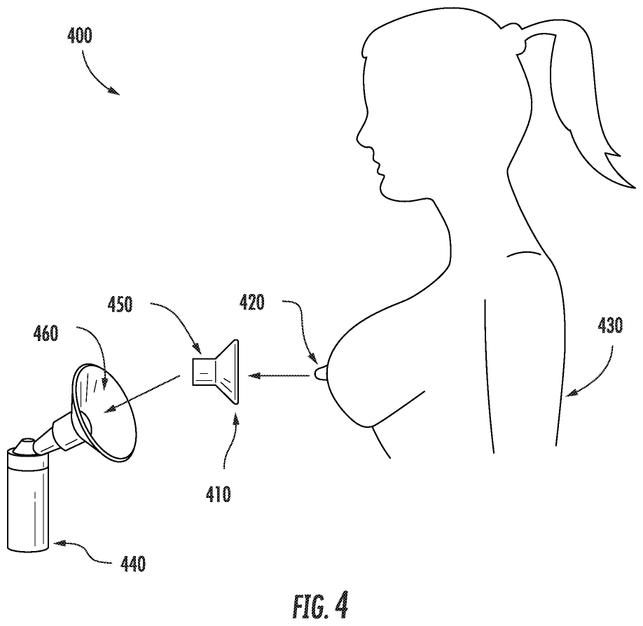

Detailed reference will now be made to embodiments of the present invention, examples of which are illustrated in FIGS. 1 through 6. In a first embodiment, and in reference to FIG. 1, there is shown a nipple cushion 100 of the present invention. Nipple cushion 100, is formed to operate in conjunction with a standard breast pump (see FIGS. 4, 440 and 450), and inserted into a breast cushion. Because breast cushions that are part of breast pumps are funnel-shaped, and are fixedly attached to a user's breast, the nipple cushion 100 of the present invention also takes the shape of a funnel.

The general funnel shape construction is preferable in order to adapt the nipple cushion for use with a breast pump and its associated breast cushion of a generally similar funnel shape. The nipple cushion includes a pump interface 105, in which on occasion, the user may fold it inwards in order to achieve better adhesion to the user's skin, and to lessen any friction resulting from the breast being moved back and forth during milking sessions. At the other end of the nipple cushion 100, is nipple and areola interface 110. An adhesive ring end 115 that may be applied around the opening of the pump interface 105 and the nipple and areola interface 110, preferably food grade and nontoxic adhesive is applied by hand or machine along 115, the outside edges or surface of the nipple cushion 100. Included in the nipple cushion 100 also is a raised section 120 which is formed around the circumference of the nipple and areola interface 110. The raised section 120 is raised on the inner part of the nipple and areola interface 110 in order to keep nipple and areola interface 110 held against the breast of the user.

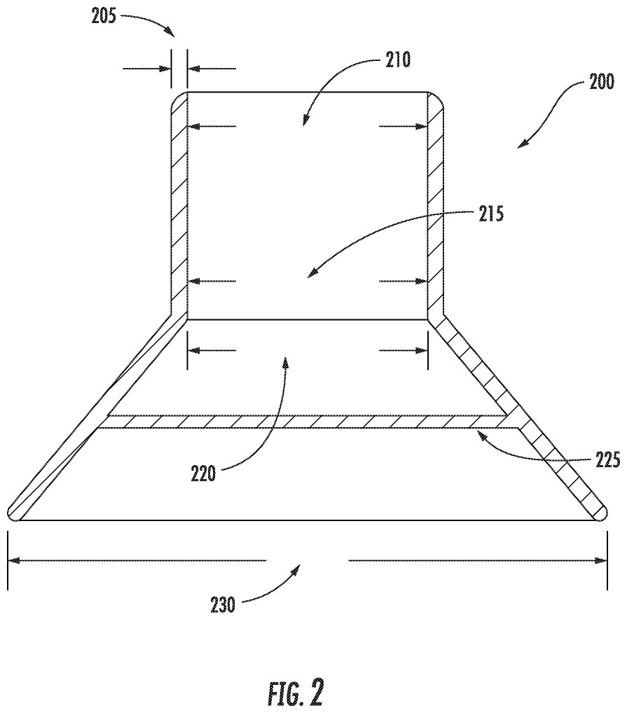

Referring now to FIG. 2, the pump interface 205 may comprise a tubular front section 210 that may be stretched into place to conform to the tunnel of the breast cushion of any breast pump, and a rear section 215 that connects a front section of the nipple and areola interface at a fuse point 220. The fuse point 220 between the pump interface 205 and the nipple and areola interface 220 proceeds to define an outward tapering funnel shape such that it terminates at the lower rim 230 of the nipple and areola interface 220. Included in the nipple cushion 200 also is a raised section 225 which is formed around the circumference of the nipple and areola interface 220. The raised section 225 is raised on the inner part of the nipple and areola interface 220 in order to keep nipple and areola interface 220 held against the breast of the user.

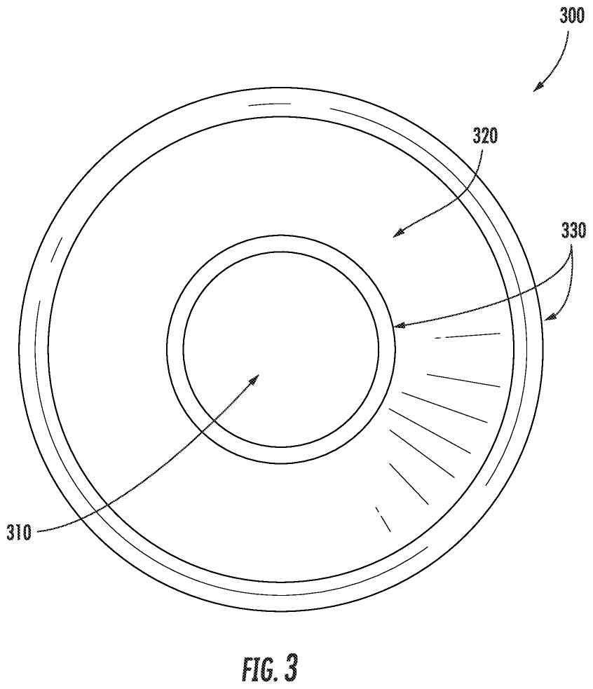

In reference to FIG. 3, there is shown a front view of the nipple cushion 300, that illustrates outer and inner rings 330, an inside view of the of the nipple and areola interface 320, and the inside tunnel of the breast pump interface 310.

In order for the present invention to conform to any size and shaped nipple as well as any size and shape breast pump cushion, the nipple cushion 200 is fabricated from an elastomeric material that is highly elastic, has high tear resistance and can exhibit both tacky and non-tacky surfaces. The present invention may be made of extremely soft and stretchy elastomeric materials to mimic the properties of human skin and to conform to any solid surface it comes in contact with under moderate to extreme pressure.

While the previous section describes the elements of the nipple cushion of the present invention, the production of the nipple cushion should yield a single unit to be used/worn by the pump user. As seen in reference to FIG. 4 as a first step, in order to achieve the desired effect, the pump interface 450 of the nipple cushion 410 should be inserted into and pressed against the channel section of the breast pump cushion 460 on the breast pump, which also consists of a hollow cone portion operatively connected to a hollow cylindrical portion. The nipple cushion 410 should preferably be inserted into the breast pump cushion 460 so as to mate the fuse point between the nipple and areola interface 410 and cylindrical pump interface in order to provide maximum comfort to the pump user. To remain securely in place and not lead to suction/vacuum pressure loss the adhesive ring 1c on both ends of the outside surfaces of the nipple cushion is preferably secured to the inside surface of the breast cushion prior to usage. If a misalignment or defective insertion of the nipple cushion is encountered after insertion into the breast cushion for breast pumping, the pump user will be able to remove the nipple cushion for breast pumping and reapply/reinstall as many times as needed to ensure a proper alignment and seal has been created.

Once inserted, the nipple and areola interface of the nipple cushion 410 is pressed in a circular motion against the inside circumference of the breast pump breast cushion 460 so that the adhesive layer (shown in FIG. 1 as 115) may bond the softer nipple cushion 410 to the more rigid breast cushion 460. The nipple areola interface 410 is pressed against the funnel shaped rear section of the breast cushion 460 in order to retain a shape that may receive the areola and nipple 420. The use of adhesives (shown in FIG. 1 as 115) to secure the rear section is recommended but not required.

The user may then insert her areola and nipple 420 into the nipple areola interface 410. Once the front and rear sections of the nipple cushion (shown in FIG. 1 as 110) are secured against the breast cushion 460, and the user has inserted her areola and nipple 420, the breast pump 440 is ready for use per the standard operating procedures for the specific breast pump 440. As will be appreciated, steps may be performed interchangeably based on the breast pump user's preference. Similarly, the usage of lubricants on the areola and nipple 420 is optional and is based on the breast pump user's preference as such. When completed, the user sets aside the breast pump milk collection units (attached to FIG. 440 the breast cushions). The nipple cushion can be left in place throughout the invention's lifespan, or can be removed to facilitate washing, used with a different set of breast cushions, or replaced with a new nipple cushion.

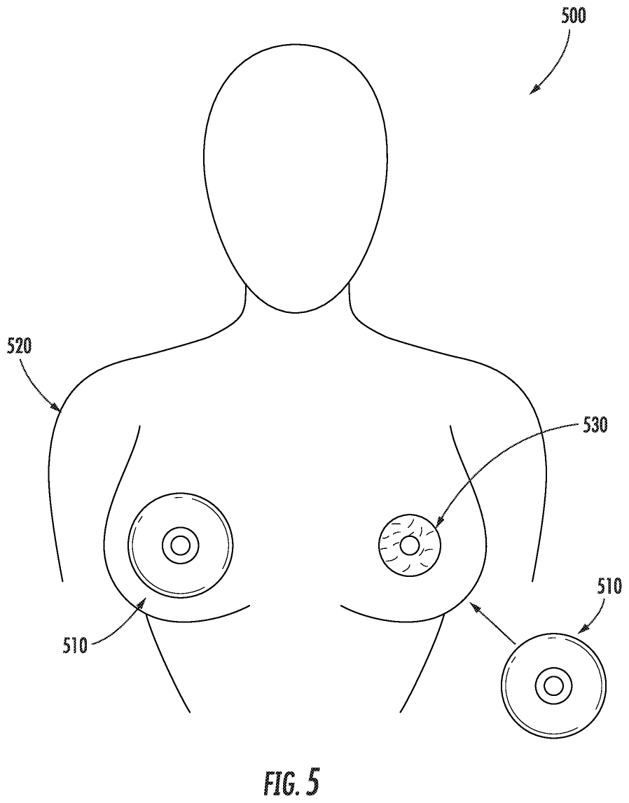

In reference to FIG. 5, there is shown a frontal view of a user 520, applying the nipple cushion 510 of the present invention. As shown in FIG. 5, the right breast of the user is having the nipple cushion 510 being applied to, while the left breast is showing the nipple and areola 530 prior to the nipple cushion 510 of the present invention having applied thereto.

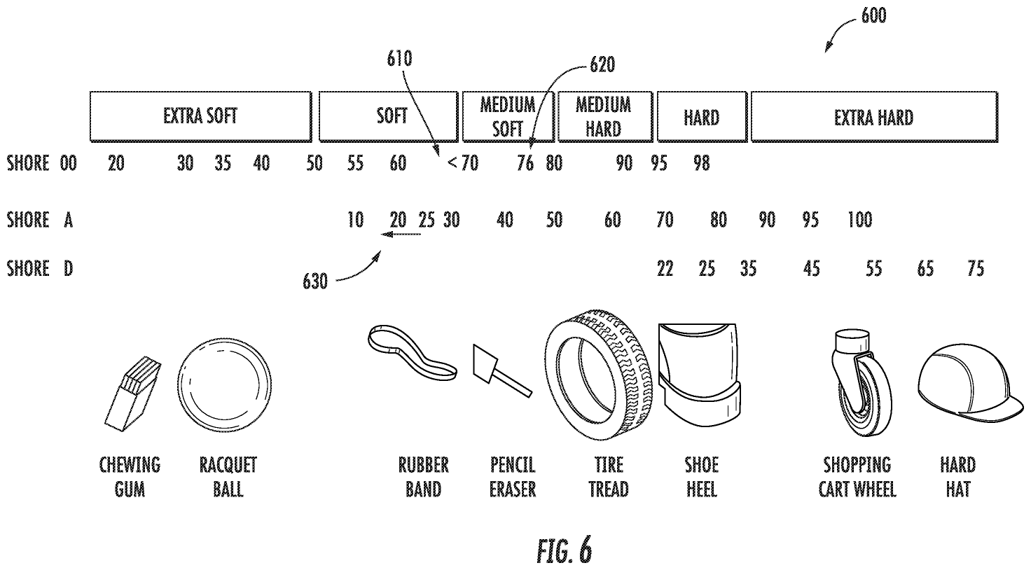

In reference to FIG. 6, there is shown a Shore table 600 scaling the hardness and softness of materials. And as indicated above, the nipple cushion (FIG. 1, reference #100) is soft (e.g. durometer rating less than Shore 25A 630, Shore 35O, or Shore 76OO 620 depending on material type) and stretchy enough (e.g. tensile elongation greater than 150% percent before break, at 73 degrees Fahrenheit) to ensure the nipple cushion can expand or be compressed to fit appropriately on any particular areola and nipple, as seen in reference to FIG. 5. Also, to prevent vacuum leakage with any breast pump cushion the present invention combines the elastic properties of the nipple cushion material with adhesives or bonding of the separate materials during the manufacturing process to form an effective seal.

Additionally, to prevent suction loss/leakage the present invention can be bound to a breast pump's breast cushion in a permanent, semi-permanent, or non-permanent manner through the application of adhesives or fusing materials during or after the manufacturing process.

As seen in reference to FIG. 1-5 a single piece with various design elements may be achieved, and any alterations would mainly be made as to the type of materials used for producing the invention, the type and location of adhesive (e.g. one, two, three, or more rings for applying the adhesive instead of two), and actual dimensions of the components. In fact, the nipple cushion 100 can take various initial shapes such as a sheet, a cylinder, a cone, a dome, or a pyramid so long as the material conforms to the surface of the nipple and areola 530 of FIG. 5, when stretched into place.

The nipple cushion 100 can be produced via hand pouring, injection molding, or additive manufacturing of materials such as thermoplastic elastomer, silicone rubber, natural rubber, latex, or other rubber-like materials. The preferred material is an injection molded thermoplastic elastomer that meets the North America and European Union food safety standards for fatty food/liquid contact and transport.

The process for injection molding thermoplastic elastomer starts with melting of pellets into a liquid form through higher than room temperature (e.g. 350 degrees Fahrenheit), then injected into a hardened steel or other solid and durable manufacturing tooling material for cast, cure or set, and removal or ejection at or below room temperature. When creating multiple units simultaneously the usage of multiple operators, or multi-cavity tooling and/or additional manufacturing lines would be required. The additional step of applying the adhesive coating 1c can either be performed by human operator or via machine after the base unit has been injection molded. In the case of additive or subtractive manufacturing a computer-aided design file is loaded into the tool (e.g. CNC, lathe, 3D printer) for cutting and/or forming into the desired shape and pattern.

Another alternative could be to design a memory material in such a way that the starting shape would differ from the components described earlier but with external catalyst (e.g. water, electrical field, etc.) the shape could be modified to resemble the invention. Soft and stretchy materials other than the elastomers mentioned could be used.

While the invention has been described by reference to certain preferred embodiments, it should be understood that numerous changes could be made within the spirit and scope of the inventive concept described. Also, the present invention is not to be limited in scope by the specific embodiments described herein. It is fully contemplated that other various embodiments of and modifications to the present invention, in addition to those described herein, will become apparent to those of ordinary skill in the art from the foregoing description and accompanying drawings. Thus, such other embodiments and modifications are intended to fall within the scope of the following appended claims. Further, although the present invention has been described herein in the context of particular embodiments, implementations and applications and in particular environments, those of ordinary skill in the art will appreciate that its usefulness is not limited thereto and that the present invention can be beneficially applied in any number of ways and environments for any number of purposes. Accordingly, the claims set forth below should be construed in view of the full breadth and spirit of the present invention as disclosed herein.

What is claimed is:

1. An apparatus comprising:
A nipple cushion for pumping breast milk from a user, comprising:
a nipple cushion being formed from an elastomer for a breast-receiving funnel and breast cushion of a breast pump;
a tubular portion of the nipple cushion that stretches and flexes with user's skin specifically the nipple;
a nipple/areola interface portion conforming to all shapes of a breast cushion belonging and use in conjunction with a breast pump; and
a stretch point located where the tubular portion and the nipple/areola interface portion meet.

2. The apparatus of claim 1, wherein the nipple cushion conforms to a funnel shape of the breast cushion of a breast pump.

3. The apparatus of claim 1, wherein the nipple cushion conforms to a user's nipple and areola.

4. The apparatus of claim 2, wherein the conforming of the nipple cushion to the generally funnel shaped breast cushion of the breast pump allows for proper suction between the user's breast and the breast pump and for the extraction of breast milk.

5. The apparatus of claim 3, wherein the nipple cushion conforms to a user's nipple and areola for reducing friction associated with a back-and-forth movement of the skin while using the breast pump for extraction of milk.

6. The apparatus of claim 4, wherein the reduction of friction averts damage to the user's skin.

7. The apparatus of claim 5, wherein the reduction of friction lessens the soreness of the user's nipple and areola associated with using a breast pump.

8. A method for facilitating pumping breast milk from a user, comprising:
forming a nipple cushion from an elastomer for a breast-receiving funnel of a breast pump;
interfacing with the breast pump a tubular portion of the nipple cushion that flexes with the user's skin;
interfacing a nipple/areola portion of the nipple cushion to conform to most shapes of a breast cushion of a breast pump; and
locating a stretch point where the tubular portion and the nipple/areola interface portion meet.

9. The method of claim 8, wherein the nipple cushion conforms to a funnel shape of a breast cushion of a breast pump.

10. The method of claim 8, wherein the nipple cushion conforms to a user's nipple and areola.

11. The method of claim 9, wherein the conforming of the nipple cushion to the funnel shaped breast cushion of the breast pump facilitates for proper suction between the user's breast and the breast pump and for letting down or extraction of breast milk.

12. The method of claim 10, wherein the nipple cushion conforms to a user's nipple and areola for reducing friction associated with a back-and-forth movement of the breast pump during milking.

13. The method of claim 11, wherein the reduction of friction and abrasion of breast and nipple tissue due to the degradation of the plastic used in manufacturing.

14. The method of claim 12, wherein the reduction of friction lessens soreness associated with friction.
